(19) United States
(12) Patent Application Publication
(10) Pub. No.: US 2024/0207602 A1
(43) Pub. Date: Jun. 27, 2024

(54) SYSTEMS AND METHODS FOR DELIVERING PHARMACEUTICAL COMPOSITIONS TO THE NASAL CAVITY USING IONTOPHORESIS

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventor: Mahyar Z. KERMANI, Austin, TX (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/555,935

(22) PCT Filed: Apr. 12, 2022
(86) PCT No.: PCT/US2022/024447

§ 371 Date: Oct. 18, 2023

Related U.S. Application Data
(60) Provisional application No. 63/185,022, filed on May 6, 2021.

(51) Int. Cl. A61N 1/04; A61K 31/137; A61K 31/167; A61K 31/573; A61N 1/05; A61N 1/30

(52) U.S. Cl. CPC A61N 1/0448; A61K 31/137; A61K 31/167; A61K 31/573; A61N 1/0546; A61N 1/306

(57) ABSTRACT

Systems and methods for delivering pharmaceutical compositions to the nasal cavity using iontophoresis. The systems and methods include a delivery system that is disposed in operative apposition with a tissue surface, which delivery system can circulate iontophoretic solution having ions of a therapeutic substance in the nasal cavity. The circulation can be continuous or periodic. The circulation can replace the iontophoretic solution within the nasal cavity over a period of time, such as from 15 seconds to 1 minute. The systems and methods further include an electrode device that is utilized to apply current to perform the iontophoresis while the circulating of the iontophoretic solution in the nasal cavity. In some embodiments, the therapeutic substance is a steroid, and in further embodiments, the ions are betamethasone ions.

### CROSS-REFERENCE TO RELATED APPLICATIONS

This application claims the benefit of U.S. Provisional Application Ser. No. 63/185,022 filed May 6, 2021, which provisional application is incorporated by reference herein as if reproduced in full below.

### TECHNICAL FIELD

Example embodiments relate to iontophoretic drug delivery systems and methods, and more particularly to systems and methods for delivering pharmaceutical compositions to the nasal cavity using iontophoresis.

### BACKGROUND

The nasal cavity forms a part of a subject's respiratory system. As shown in FIGS. 1A-1B, the nasal cavity 101 is an air-filled space disposed behind an exterior portion of the subject's nose. Four pairs of sinuses are disposed around the nasal cavity, including the ethmoid sinuses 102, the maxillary sinuses 103, the frontal sinuses 104, and the sphenoid sinuses 105. The ethmoid sinuses 102 are located in the ethmoid bone, which separates the nasal cavity from the brain, the maxillary sinuses 103 are located behind the cheekbones near the maxillae or upper jaws, the frontal sinuses 104 are located in the center of the frontal bone or forehead above each eye, and the sphenoid sinuses 105 are located in the sphenoid bone near the optic nerve and the pituitary gland.

The nasal cavity is divided by a vertical partition, the nasal septum 106, into a right and a left side. Both sides of the nasal cavity 101 are hollow and normally filled with air. The nasal cavity 101 is exposed to the atmosphere of the outside environment via the anterior nares 107 of the nose, as shown in FIG. 1. The anterior nares 107 allow for the inhalation and exhalation of air through the nasal cavity 101. The nasal cavity 101 is bounded by sidewalls, which include three pairs of turbinates or nasal concha, namely, the inferior turbinates 108, the middle turbinates 109, and the superior turbinates 110. The turbinates 108-110 project into the nasal cavity 101 and divide the nasal passage of the nasal cavity 101 into four groove-like air passages. As shown in FIG. 1, the inferior turbinates 108 are disposed below the middle turbinates 109 and the superior turbinates 110 on the nasal septum 106, the middle turbinates 109 are disposed between the inferior turbinates 108 and the superior turbinates 110, and the superior turbinates 110 are disposed above the middle turbinates 109 and the inferior turbinates 108. The turbinates 108-110 are responsible for regulating airflow during inhalation.

The nasal cavity opens into the nasopharynx 111, which forms the upper part of the pharynx or throat. The nasopharynx 111 contains a collection of lymphoid tissue towards the midline known as adenoids 112. The nasopharynx 111 also includes a Eustachian tube opening 113, which connects the nasopharynx 111 to the middle ear via the Eustachian tube. The Eustachian tube serves as an air channel between the middle ear and the nasopharynx 111 that helps fill the middle ear with air and equalize the air pressure of the middle ear with the atmosphere.

The nasal cavity 101 and the sinuses 102-105 are lined with tissue known as mucosa that produces mucus. The mucus-covered surfaces of the nasal cavity 101 help filter, humidify, and warm or cool air that is inhaled by a subject. The mucus-covered surfaces also trap harmful particles such as allergens or bacteria. The nasal cavity 101 and its surrounding tissue, however, can become inflamed, infected, or obstructed. To treat these conditions, a physician may need to deliver therapeutic substances to the nasal cavity or its surrounding tissue. For example, a physician can deliver a therapeutic substance such as an anesthetic to tissue surrounding or proximate to the nasal cavity 101 to alleviate pain or other discomfort during a surgical operation (e.g., a skull-based surgery, septoplasty, dental surgery, etc.). The physician can also delivery other therapeutic substances (e.g., analgesics, anti-inflammatoires, antibiotics, antivirals, antifungals, antiparasitics, decongestants, mucokinetics, antihistamines, antioxidants, immunosuppressive agents, dissociatives, steroids, sedatives or hypnotics, anticholinergics, antiemetics, antiepiletics, non-steroidal anti-inflammatory drugs (NSAIDs), etc.) to reduce or treat inflammation, infection, congestion, pressure, and/or other conditions within the nasal cavity and/or other conditions of a patient's body.

Delivery of therapeutic substances, including anesthetics, to the nasal cavity 101 or surrounding tissue regions can be used to treat and/or aid in treatment of allergic or non-allergic rhinitis, nasal obstruction (e.g., an obstruction caused by sinusitis, allergies, etc. or an anatomical factor such as a deviated septum, enlarged adenoids, nasal polyps, foreign objects, turbinate hypertrophy, nasal valve collapse, etc.), nasal polyps, sinusitis (e.g., ostium, intra-sinus, post-sinus surgery), epistaxis, allergies, migraines, and tinnitus, polyposis, etc.

Oftentimes, the therapeutic substance may need to be maintained against a tissue surface for an extended period of time to provide effective results. For example, for treating inflammation or applying an anesthetic, a therapeutic substance may need to be applied to a tissue surface within the nasal cavity for a sufficient period of time such that the therapeutic substance can diffuse or perfuse through the surface into the tissue. U.S. Patent Appl. Publ. No. 2020/00276434, filed Apr. 7, 2020, (“the '434 patent application”) (which is attached hereto at Appendix A) is directed to systems, apparatus, and methods for delivering a therapeutic substance to a target area within or proximate to a nasal cavity of a subject using iontophoresis and/or electroosmosis. Iontophoresis is a method for delivering a drug across a biological membrane, such as the skin, the tympanic membrane (for the ear), and the mucosa (for the nasal cavity). By applying low-level electrical current to a similarly charged drug solution, iontophoresis repels ions of the drug, thus transporting them across the skin or other membrane. Molecules are transported across the stratum corneum by electrophoresis and electroosmosis and the electric field can also increase the permeability of the skin. These phenomena, directly and indirectly, constitute active transport of matter due to an applied electric current.

Additional systems and methods for delivering a therapeutic substance to the nasal cavity or surrounding tissue are desirable.

### SUMMARY

The present invention relates to systems and methods for delivering pharmaceutical compositions to the nasal cavity using iontophoresis.

In general, in one aspect, the invention features a system that includes a reservoir structure operable for receiving an iontophoretic solution including ions of a therapeutic substance. The system further includes a delivery system including a delivery interface configured to be placed in operative apposition with a first side of a tissue surface within a nasal cavity of a subject. The delivery system is configured for circulating the iontophoretic solution to the reservoir structure over a predefined period of time. The system further includes an electrode device including a proximal portion and a distal portion. The electrode device configured for the proximal portion to be disposed outside of the nasal cavity while the distal portion extends into the nasal cavity into engagement with a portion of the reservoir structure. The electrode device further configured for applying a current to the reservoir structure such that an amount of the ions of the therapeutic substance is delivered to a target area of the subject on a second side of the tissue surface. The reservoir structure configured to maintain the delivery interface against the first side of the tissue surface for at least the predefined period of time during which current is being applied by the electrode device to the reservoir structure. The circulating of the iontophoretic solution replenishes concentration of the ions of the therapeutic substance within the reservoir structure during the predefined period of time to enhance amounts of the therapeutic substance that are delivered to the target area.

Implementations of the invention can include one or more of the following features:

The delivery system can be configured for circulating the iontophoretic solution having a continuous flow of iontophoretic solution.

The delivery system can be configured for circulating the iontophoretic solution having a periodic flow of iontophoretic solution.

The delivery system can be configured for circulating the iontophoretic solution at an average flow rate that replaces the iontophoretic solution in the reservoir structure in a period between 15 seconds and 1 minute.

The delivery system can be configured to provide for the iontophoretic solution to flow into the reservoir structure at or nearby the tissue surface. The delivery system can be configured to provide for the iontophoretic solution to flow away from the reservoir structure at or nearby the distal portion of the electrode device.

The electrode device can be configured for applying the current utilizing a current profile having a ramp-up period, a steady state period, and a ramp-down period.

The delivery system can be configured for circulating the iontophoretic solution during the steady state period and for not circulating the iontophoretic solution during the ramp-up period and during the ramp-down period.

The delivery system can be configured for circulating the iontophoretic solution during the ramp-up period at an average ramp-up period flowrate. The delivery system can be configured for circulating the iontophoretic solution during the steady state period at an average steady state period flow rate. The average steady state period flow rate can be greater than the average ramp-up period flowrate.

The system can further include a deployment device that is capable of being coupled and uncoupled to the delivery system. The deployment device can be operable for placing the delivery system within the nasal cavity.

The system can further include a retrieval device that is capable of being coupled and uncoupled to the delivery system. The retrieval device can be operable for removing the delivery system from the nasal cavity.

The system can further include a deployment/retrieval device that is capable of being coupled and uncoupled to the delivery system. The deployment/retrieval device can be operable for placing the delivery system within the nasal cavity and is further operable for removing the delivery system from the nasal cavity:

The therapeutic substance can be selected from a group consisting of analgesics, anesthetics, anti-inflammatoires, antibiotics, antivirals, antifungals, antiparasitics, decongestants, mucokinetics, antihistamines, antioxidants, immunosuppressive agents, dissociatives, steroids, sedatives, hypnotics, anticholinergics, antiemetics, antiepiletics, non-steroidal anti-inflammatory drugs, and combinations thereof.

The therapeutic substance can be a steroid.

The iontophoretic solution can include betamethasone ions.

The therapeutic substance can be an anesthetic.

The iontophoretic solution can include lidocaine ions, epinephrine ions, or a combination thereof.

In general, in another aspect, the invention features a method that includes disposing a delivery system including a delivery interface in a nasal cavity of a subject such that the delivery interface is in operative apposition with a tissue surface. The method further includes utilizing the delivery system to circulate an iontophoretic solution including ions of a therapeutic substance into the nasal cavity. The method further includes, while circulating the iontophoretic solution utilizing the delivery system, applying, during a predefined period of time and using an electrode device, a current to the delivery system such that a therapeutically effective dose of the therapeutic substance is delivered via the delivery interface to a target area below the tissue surface. The circulating of the iontophoretic solution replenishes concentration of the ions of the therapeutic substance within the nasal cavity during the predefined period of time to enhance amounts of the therapeutic substance that are delivered to the target area.

Implementations of the invention can include one or more of the following features:

The step of circulating the iontophoretic solution can be a continuous flow of iontophoretic solution.

The step of circulating the iontophoretic solution can be a periodic flow of iontophoretic solution.

The step of circulating the iontophoretic solution can be at an average flow rate that replaces the iontophoretic solution in the nasal cavity in a period between 15 seconds and 1 minute.

The step of circulating the iontophoretic solution can include flowing the iontophoretic solution into the nasal cavity at or nearby the tissue surface. The step of circulating the iontophoretic solution can include flowing the iontophoretic solution at or nearby the electrode device away from the nasal cavity.

The step of applying the current to the delivery system can include applying the current utilizing a current profile having a ramp-up period, a steady state period, and a ramp-down period.

The step of circulating the iontophoretic solution can include circulating the iontophoretic solution during the steady state period and not circulating the iontophoretic solution during the ramp-up period and during the ramp-down period.

The step of circulating the iontophoretic solution can include circulating the iontophoretic solution during the ramp-up period at an average ramp-up period flowrate. The step of circulating the iontophoretic solution can include circulating the iontophoretic solution during the steady state period at an average steady state period flow rate. The average steady state period flow rate can be greater than the average ramp-up period flowrate.

The method can further include removing the delivery system from the nasal cavity.

The method can further include performing a test to determine efficacy of the therapeutic substance delivered to the target area.

The therapeutic substance can be selected from a group consisting of analgesics, anesthetics, anti-inflammatoires, antibiotics, antivirals, antifungals, antiparasitics, decongestants, mucokinetics, antihistamines, antioxidants, immunosuppressive agents, dissociatives, steroids, sedatives, hypnotics, anticholinergics, antiemetics, antiepiletics, non-steroidal anti-inflammatory drugs, and combinations thereof.

The therapeutic substance can be a steroid.

The iontophoretic solution can include betamethasone ions.

The therapeutic substance can be an anesthetic.

The iontophoretic solution can include lidocaine ions, epinephrine ions, or a combination thereof.

In general, in another aspect, the invention features a system that includes a reservoir structure operable for receiving an iontophoretic solution including ions of a therapeutic substance. The system further includes a delivery system including a delivery interface configured to be placed in operative apposition with a first side of a tissue surface within an interior region of a subject. The delivery system is configured for circulating the iontophoretic solution to the reservoir structure over a predefined period of time. The system further includes an electrode device including a proximal portion and a distal portion. The electrode device configured for the proximal portion to be disposed outside of the interior region while the distal portion extends into the interior region into engagement with a portion of the reservoir structure. The electrode device further configured for applying a current to the reservoir structure such that an amount of the ions of the therapeutic substance is delivered to a target area of the subject on a second side of the tissue surface. The reservoir structure configured to maintain the delivery interface against the first side of the tissue surface for at least the predefined period of time during which current is being applied by the electrode device to the reservoir structure. The circulating of the iontophoretic solution replenishes concentration of the ions of the therapeutic substance within the reservoir structure during the predefined period of time to enhance amounts of the therapeutic substance that are delivered to the target area.

In general, in another aspect, the invention features a method that includes disposing a delivery system including a delivery interface in an interior region of a subject such that the delivery interface is in operative apposition with a tissue surface. The method further includes utilizing the delivery system to circulate an iontophoretic solution including ions of a therapeutic substance into the interior region. The method further includes, while circulating the iontophoretic solution utilizing the delivery system, applying, during a predefined period of time and using an electrode device, a current to the delivery system such that a therapeutically effective dose of the therapeutic substance is delivered via the delivery interface to a target area below the tissue surface. The circulating of the iontophoretic solution replenishes concentration of the ions of the therapeutic substance within the interior region during the predefined period of time to enhance amounts of the therapeutic substance that are delivered to the target area.

### DESCRIPTION OF DRAWINGS

FIGS. 1A-1B are front and side views illustrating the anatomy of a nasal cavity and surrounding regions.

FIG. 2 is a schematic of the use of a pharmaceutical composition in the iontophoresis system.

FIG. 3 is a graph showing a current profile that can provide a pre-determined measured dosage of the active ingredient in an iontophoresis system shown in FIG. 2.

FIGS. 4A-4B are graphs showing the effects of dose drug delivery using the iontophoresis system shown in FIG. 2 due to the various factors.

FIG. 5 is a schematic of an embodiment of the present invention using a pharmaceutical composition with replenishment of the pharmaceutical composition in an iontophoresis system.

FIGS. 6A-6B are graphs showing the effects of dose drug delivery due to the embodiment of the present invention shown in FIG. 5 (as compared to the ideal and non-ideal curves of FIGS. 4A-4B).

FIG. 7 is a block diagram of an embodiment of a system of the present invention positioned in a nasal cavity.

FIG. 8 is a flow diagram illustrating a method for delivering a therapeutic substance to a target area in a nasal cavity of a subject in accordance with some embodiments of the present invention.

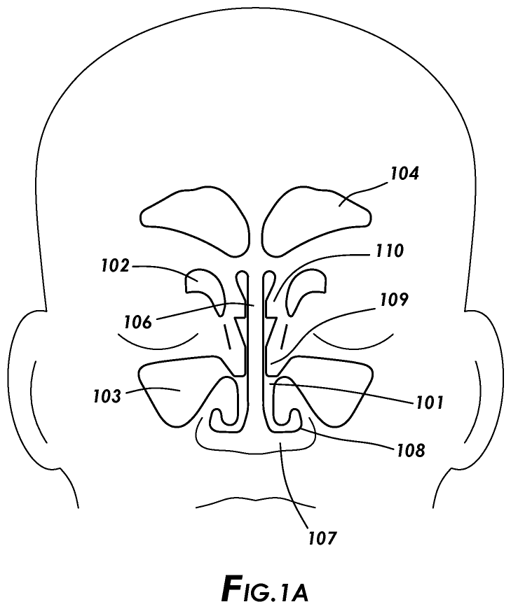

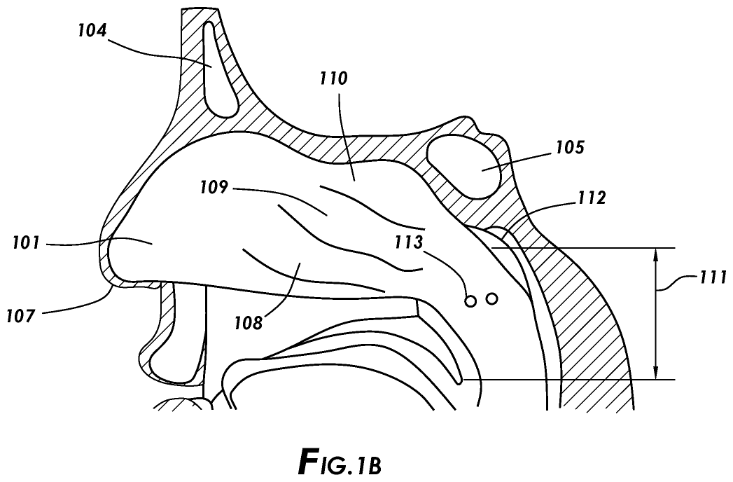

### DETAILED DESCRIPTION

Example embodiments relate to iontophoretic drug delivery systems and methods, and more particularly to systems and methods for delivering pharmaceutical compositions to the nasal cavity using iontophoresis Therapeutically, electromotive drug administration (EMDA) delivers a medicine or other chemical through the skin or other tissue surfaces. It is different from dermal patches, which do not rely on an electric field. Iontophoresis drives a charged substance, usually a medication or bioactive agent, by repulsive electromotive force, through the skin or other tissue surfaces. A small electric current is applied to an iontophoretic reservoir structure placed on the skin or other tissue surface, containing a charged active agent and its solvent vehicle. Another chamber or a skin/other tissue electrode carries the return current. The positively charged chamber, called the anode, will repel a positively-charged chemical species, whereas the negatively charged chamber, called the cathode, will repel a negatively-charged species into the skin.

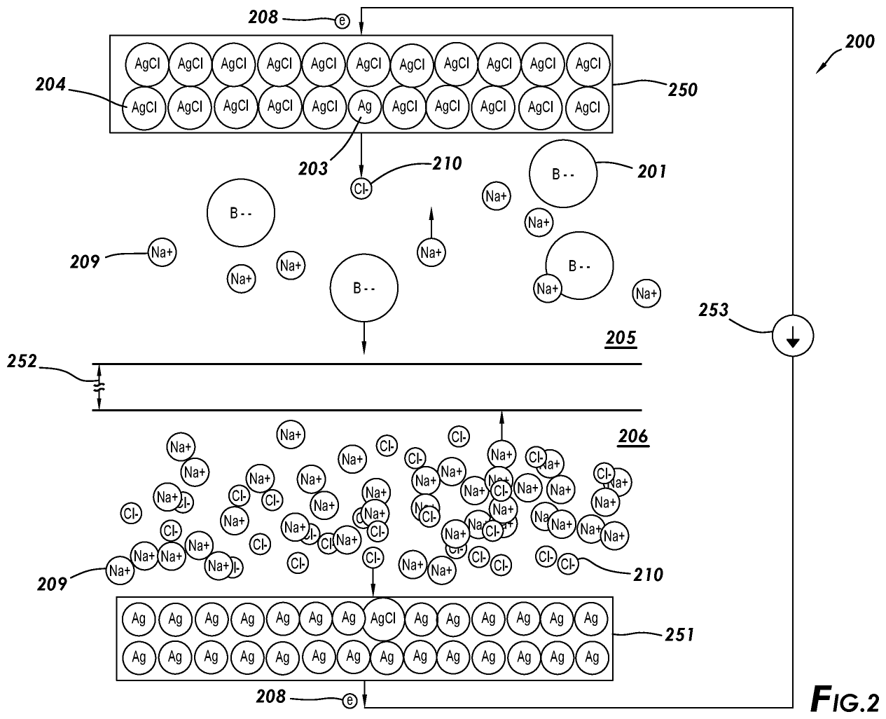

FIG. 2 is a schematic showing an iontophoresis system 200 having a pharmaceutical composition that includes negative ions of the active ingredient (betamethasone ions (B⁻²) 201. As seen in FIG. 2, cathode 250 includes Ag 203 and AgCl 204 and is positioned on the drug (or donor) side 205 of mucosal tissue 252. Anode 251 includes Ag 203 and AgCl 204 and is positioned on the receptor side 206 (the body side) of mucosal tissue 252.

The chemical formula for B⁻² 201 is C₂₂H₂₈FO₈P⁻². B⁻² 201 are ions in the pharmaceutical composition by dissociation in water, such as, typically, by the dissociation of betamethasone sodium phosphate (C₂₂H₂₈FNa₂O₈P) per the formula:

C₂₂H₂₈FNa₂O₈P→C₂₂H₂₈FO₈P⁻²+2Na⁺ (1)

Accordingly, the pharmaceutical composition will also have the non-active ingredient sodium ions (Na⁺) 209 due to this dissociation. Moreover, in some pharmaceutical compositions there can be sodium chloride (NaCl), which are non-active ingredients (that disassociate to Na⁺ 209 and Cl⁻ 210).

In an iontophoresis system 200, control unit 253 is used as the charger for cathode 250 and anode 251. Control unit 253 provides the small electric current (from an external generator), which permits the flow of electrons 208 from anode 251 to cathode 250. At anode 251, the electrons are generated as follows:

$${Ag}^{+} + {Cl}^{-} \to AgCl + e \qquad ( 2 )$$

At cathode 250, the electrons are combined as follows:

$$e + AgCl \to {Ag}^{+} + {Cl}^{-} \qquad ( 3 )$$

When control unit 253 is providing the electric current, cathode 250 is a negatively-charged cathode (which repeals the negatively-charged chemical species, i.e., B⁻² 201 and Cl— 210) and anode 251 is a positively-charged anode (which attracts the negatively-charged chemical species). This results in the negatively-charged chemical species being driven through mucosal tissue 252. By controlling the electric current of the control unit 253, this controls the driving of the negatively-charged chemical species.

It should be noted that if the chemical species to be driven through mucosal tissue 252 is a positively-charged species, then the positions of cathode 250 and anode 251 in iontophoresis system 200 would need to be reversed, i.e., cathode 250 would be on receptor side 206 and anode 251 would be on drug side 205.

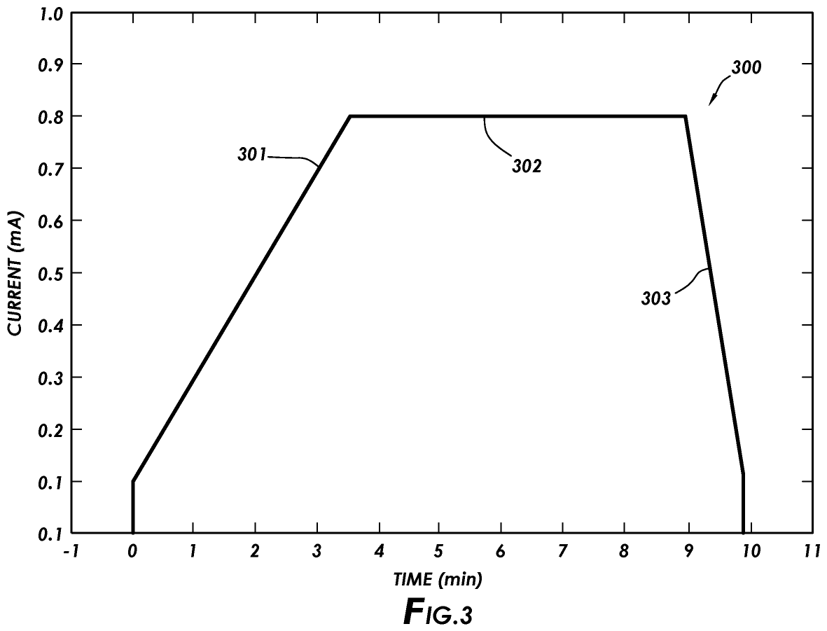

FIG. 3 shows a representative example of a current profile that can provide a pre-determined measured dosage of the active ingredient (B⁻² 201) in iontophoresis system 200. The current profile has three sections, namely, a first section 301 in which the current of current profile 300 is ramped-up over a period of time. As shown in current profile 300, during the first section 301 (the “ramp-up period”), the current is ramped-up from 0.1 mA to 0.8 mA for a time period of about 3.5 minutes (beginning at time zero and ending at about time 3.5 minutes during the overall procedure time of current profile 300). This calculates to a “ramp-up rate” of around 3.33 μA/sec in current profile 300. The “ramp-up rate” is the change of current over the ramp-up time.

During the second section 302 (the “steady state period”), the current is maintained at the predetermined current (“steady state current”) utilized for the treatment, which, in current profile 300, is a steady state current of 0.8 mA for a time period of about 5.5 minutes (beginning at about time 3.5 minutes and ending at about time 9 minutes during the overall procedure time of current profile 300).

During the third section 303 (the “ramp-down period”), the current is ramped-down from 0.8 mA at a rate of 12.3 uA/sec, which takes about 1 minute (beginning at about time 9 minutes ending at about time 10 minutes during the overall procedure time of current profile 300).

The sum of sections 301-303 yields an overall time period (the “application time”) of current profile 300 of about 10 minutes.

As iontophoresis occurs, there are several factors that have been discovered that significantly reduce the delivery of B⁻² 201 across the mucosal tissue 252 to the body side 206 for delivery to the subject. These factors include:
(a) The concentration of Cl⁻ 209 is increasing on the donor side (due to generation of Cl⁻ 209 per Equation (3)).
(b) The concentration of B⁻² 201 is decreasing on the donor side (due to B⁻² 201 leaving the donor side across mucosal tissue 252); and
(c) The concentration of Na⁺ 210 is increasing on the donor side (due to Na⁺ 210 entering the donor side across mucosal tissue 252).

Cl⁻ 209 and Na⁺ 210 are competing ions in the iontophoresis system. As used herein, “competing ions” are ions present in the pharmaceutical composition that are not ions of the active ingredient in the pharmaceutical composition but are ions that compete against the delivery of the ions of the active ingredient species.

The competing ions can have the same directional charge (positive or negative) as the charge of the ions of the active ingredient in the pharmaceutical composition. The magnitude of the charge of the competing ion can be the same or different from the ions of the active ingredient. I.e., the active ingredient ion could have a negative 2 (−2) charge (such as B⁻²), while the same-directional competing ion could have a negative 1 (−1) charge (such as Cl⁻). In such instance, the same-directional competing ions are attracted to and repulsed by the cathode and anode in the same direction as the active ingredient ions such that active ingredient ions and the non-active ingredient ions are both driven by the iontophoresis system in the same direction across the mucosal tissue.

For example, when utilizing an iontophoresis system in which the pharmaceutical composition has ions of active ingredients that are negatively-charged species (such as B⁻²), Cl⁻ are same-directional competing ions. In such circumstance, the presence and concentration of Cl⁻ effects the driving of the active ingredients across the mucosal tissue, as some of the current is utilize on the Cl⁻.

The competing ions can have the opposite directional charged ions as the charge of the ions of the active ingredient in the pharmaceutical composition. For example. Na⁺ are also a competing ion against delivery of the negatively-charged species (such as B⁻²).

Likewise, when utilizing an iontophoresis system in which the pharmaceutical composition has ions of active ingredients that are positively-charged species, Na⁺ and Cl⁻ are competing ions. In such circumstance, the presence and concentration of Na⁺ and Cl⁻ effects the driving of the active ingredients across the mucosal tissue.

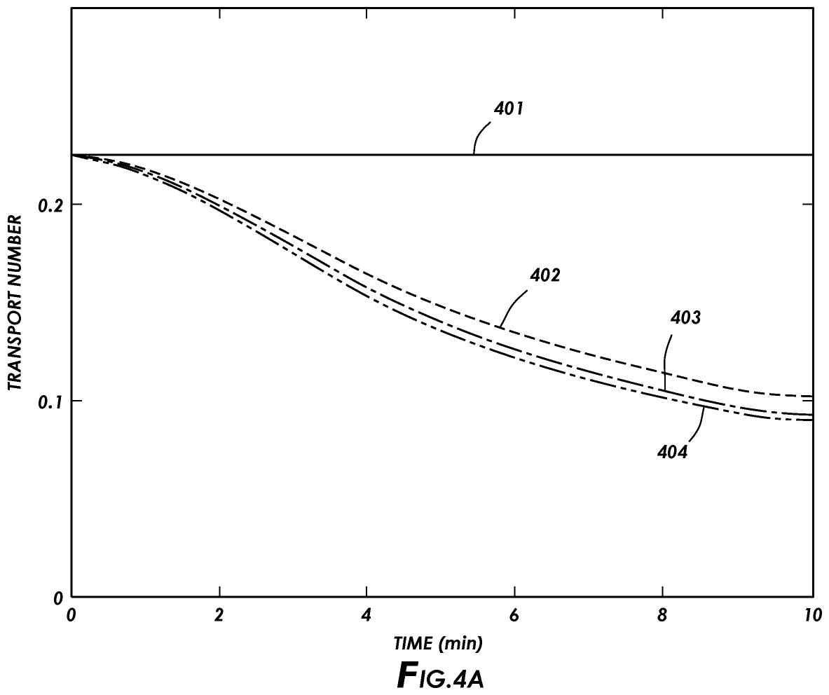

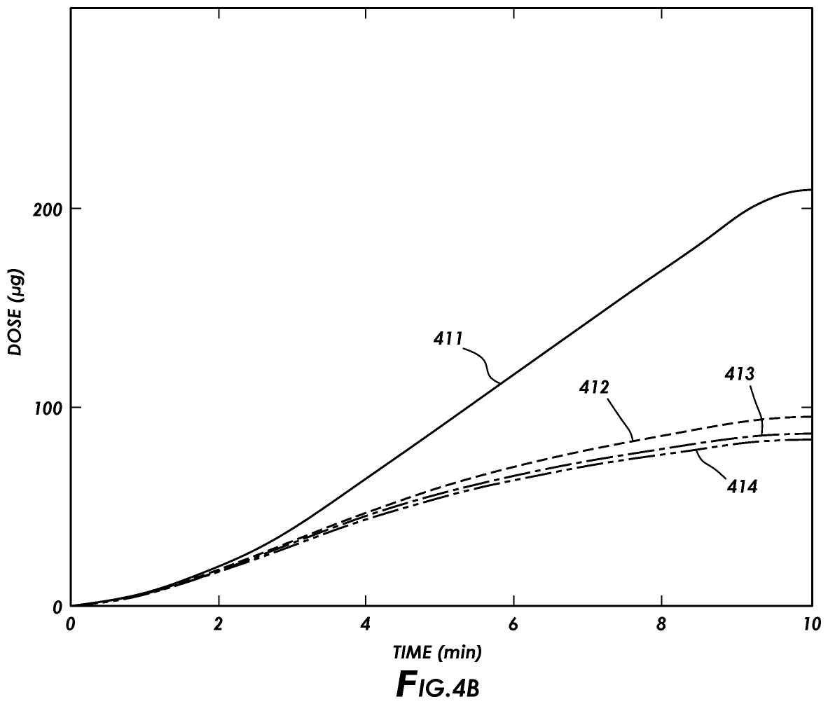

FIGS. 4A-4B shows the effects of dose drug delivery due to the various factors. Curves 401-404 of FIG. 4A reflect the transport number of the active ingredient (B⁻²) over time. Curve 401 is the ideal case. Curve 402 is the non-ideal case due to increased concentration of Cl⁻ on the donor side. Curve 403 is the non-ideal case due to increased concentration of Cl⁻ on the donor side and decreased concentration of B⁻² on the donor side. Curve 404 is the non-ideal case due to increased concentration of Cl⁻ on the donor side, decreased concentration of B⁻² on the donor side, and increased concentration of Na⁺ on the donor side. (As shown by a comparison of curves 402-404, the impact of increased concentration of Cl⁻ on the donor side is greater than the impacts of each of the decreased concentration of B⁻² on the donor side and the increased concentration of Na⁺ on the donor side).

FIG. 4B shows the dosage drug delivery that is provided using the iontophoresis system 200 when applying current profile 300. Curves 411-414 of FIG. 4B reflect the cumulative dosage drug delivery of the active ingredient (B⁻²) over time that is provided using the iontophoresis system 200 when applying current profile 300. Curve 411 is the ideal case of drug delivery. Curve 412 is the non-ideal case due to increased concentration of Cl⁻ on the donor side. Curve 413 is the non-ideal case due to increased concentration of Cl⁻ on the donor side and decreased concentration of B⁻² on the donor side. Curve 414 is the non-ideal case due to increased concentration of Cl⁻ on the donor side, decreased concentration of B⁻² on the donor side, and increased concentration of Na⁺ on the donor side. (As shown by a comparison of curves 412-414, the impact of increased concentration of Cl⁻ on the donor side is greater than the impacts of each of the decreased concentration of B⁻² on the donor side and the increased concentration of Na⁺ on the donor side).

As shown in FIG. 4B, the non-ideal cases can be less than half of the ideal case. In view of the discovery of the impact of these factors on drug delivery, an improved iontophoresis system has been developed to maintain better the conditions closer to ideal so as to reduce (or eliminate) these factors (and these factors impacts).

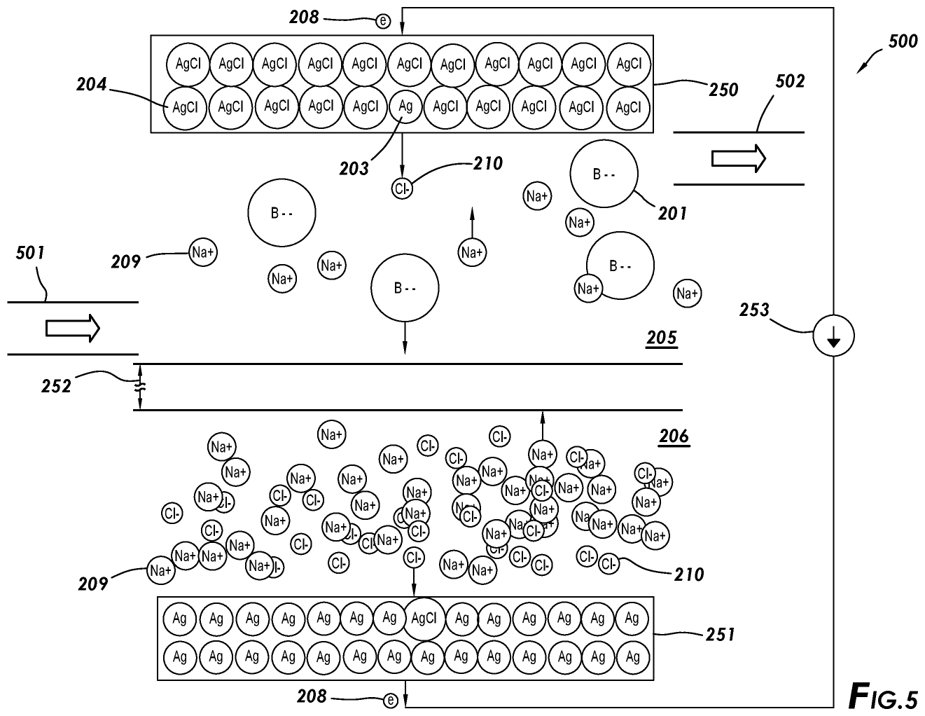

FIG. 5 is a schematic of an embodiment of the present invention showing an iontophoresis system 500 having a pharmaceutical composition that includes negative ions of the active ingredient (betamethasone ions (B⁻²) 201. As show in FIG. 5, the pharmaceutical composition is being refreshed during the process by pumping the pharmaceutical composition at flow 501 near mucosal tissue 252 and then removing the pharmaceutical composition at flow 502 near the electrode of the cathode 250. Such flow of the pharmaceutical composition can be replenished periodically or circulated constantly to enhance the dosage drug delivery. The periodic replenishment (or refreshment) can be a series of pulsed flows over a short time durations. The average flow rate of the pharmaceutical composition can be such that the pharmaceutical composition is replenished over a predetermined time in the range from 15 seconds to 60 seconds, such as every 30 seconds.

By this flow of the pharmaceutical composition, the dosage drug delivery becomes much closer to the ideal case and the various factors (and these factors' impacts) are significantly reduced (or eliminated). This is because the flow of the pharmaceutical composition serves to dampen the increase of concentrations of Cl⁻ 209 Na⁺ 210 on the donor and also serves to maintain the concentrations of B⁻² 201 on the donor side (i.e., lessens the decrease of concentration of B⁻² 201 as B⁻² leaving the donor side across mucosal tissue 252).

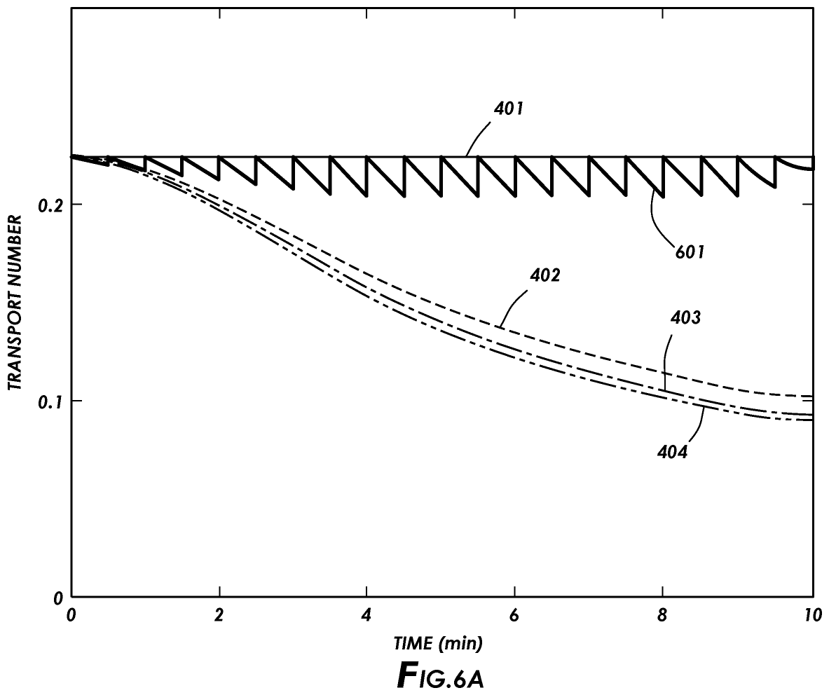

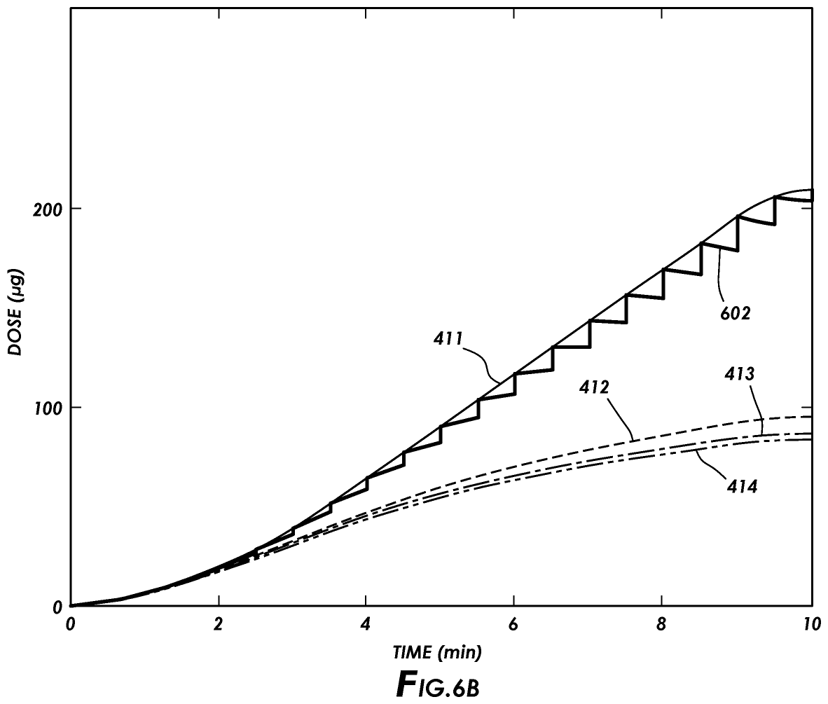

FIGS. 6A-6B shows the effects of dose drug delivery due to an embodiment of the present invention as compared with the ideal and non-ideal curves previously shown in FIGS. 4A-4B. In this embodiment, the pharmaceutical composition was replaced over a predetermined time of 30 seconds. As used herein, a pharmaceutical composition is “replaced” over a predetermined time when the average flow rate (over the predetermined time) multiplied by the predetermined time equals the cell volume of the pharmaceutical composition on the donor side. By way of example, for a cell volume of 0.5 ml of pharmaceutical composition to be replaced in 30 seconds, this would be an average flow rate of 1 mL/min. If the cell volume were 1 mL, then the average flow rate would be 2 mL/min to replace the pharmaceutical composition in the cell.

Curve 601 of FIG. 6A reflects the transport number of the active ingredient (B⁻²) over time. With the fixed replacement (or replenishment) time of 30 seconds, the transport number deviation from the ideal case (curve 401) depends on the electrical charge. Curve 602 of FIG. 6B reflects the cumulative dosage drug delivery of the active ingredient (B⁻²) over time. By replenishing the pharmaceutical composition every 30 seconds, the derivation from the ideal case would be only around 2.5%, which is significantly less than the more than 50% deviations resulting from the non-ideal cases shown by curves 412-414. Calculations reflect that by not replenishing the pharmaceutical composition, the deviation from the ideal case (curve 411) would be a little over 60%.

During ramp-up period and ramp-down periods, the derivations due to the various factors is less. Therefore, the flow for replenishing the pharmaceutical composition can be adaptively adjusted to the current profile without much degradation in transport number. This means that the flow of the pharmaceutical composition can be dynamically adjusted with the current profile.

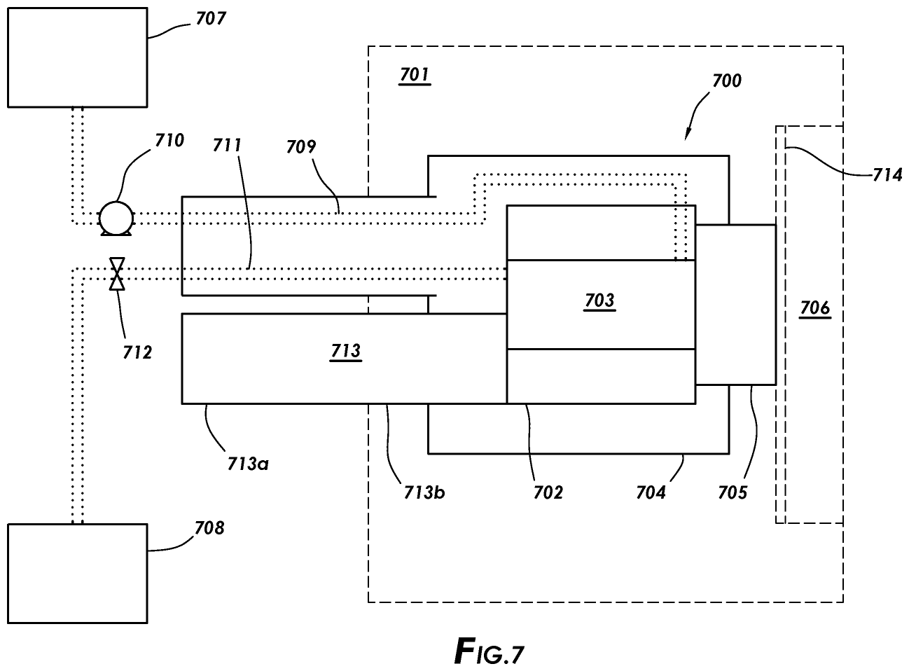

FIG. 7 shows a diagram of iontophoretic drug delivery system 700 positioned in nasal cavity 701. System 700 includes a reservoir structure 702 that can receive a volume of iontophoretic solution 703 (which contains ions of a therapeutic substance, like B⁻²). The system 700 further includes a delivery system 704 that includes a delivery interface 705. As shown in FIG. 7, the delivery system 704 can be positioned so that that the delivery interface 705 is in apposition with a tissue surface 714 with target area 706 for the iontophoresis on the other side of tissue surface 714.

There is a supply 707 that contains iontophoretic solution 703 that can be flowed through conduit 709 (such as by utilizing pump 710 or other equipment) so that the fresh iontophoretic solution 703 can be replenished in reservoir structure 702. The flow of iontophoretic solution 703 into reservoir structure 702 can be at, or nearby, tissue surface 714. There is also a drain 708 in which utilized iontophoretic solution 703 can flowed out of reservoir structure 702 through conduit 711 (such as by utilizing a valve 712 or other equipment). Generally, the supply 707 and drain 708 are positioned outside the nasal cavity.

The system further includes an electrode device 713 that has both a proximal portion 713a and a distal portion 713b. As shown in FIG. 7, proximal portion 713a can be disposed outside of nasal cavity 701 while distal portion 713b extends into nasal cavity 701 into engagement with a portion of reservoir structure 702. While electrode device 713 can be separable or integral with delivery system 704. The flow of utilized iontophoretic solution 703 from reservoir structure 702 can be at, or nearby, the drug side electrode of the electrode device 713.

Electrode device 713 is configured to apply a current to reservoir structure 702 such that an amount of the ions of the therapeutic substance in iontophoretic solution 703 is delivered to a target area 706 of the subject on the other side of tissue surface 714. Such arrangement can be similar to iontophoresis system 500 shown in FIG. 5.

Reservoir structure 702 can maintain delivery interface 705 against the tissue surface 714 for at least the predefined period of time during which current is being applied by electrode device 713 to reservoir structure 702.

The flow (i.e., circulation) of the iontophoretic solution 703 replenishes concentration of the ions of the therapeutic substance within the reservoir structure 702 during the predefined period of time to enhance amounts of the therapeutic substance that are delivered to the target area 706.

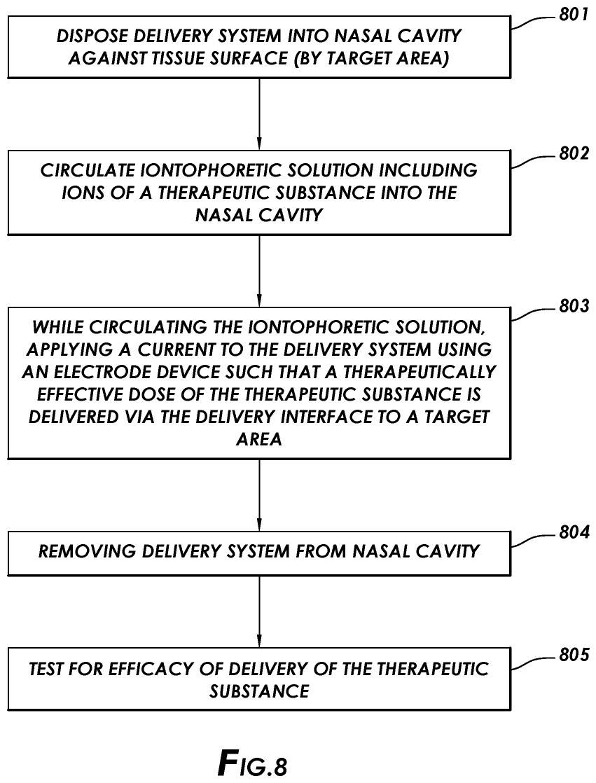

FIG. 8 is a flow diagram illustrating a method for delivering a therapeutic substance to a target area in a nasal cavity of a subject, such as can by using iontophoretic drug delivery system 700.

In step 801, the method includes disposing the delivery system (including the delivery interface) in the nasal cavity of a subject such that the delivery interface is in operative apposition with a tissue surface (by the target area). The delivery system can be disposed by using a deployment device, which can be a single component or mechanism such as, for example, a flexible shaft, beam, bar, wire, etc. The deployment device can be capable of being coupled and uncoupled to the delivery system for disposing of the delivery system.

In step 802, the method further utilizing the delivery system to circulate an iontophoretic solution including ions of a therapeutic substance (such as B⁻²) into the nasal cavity:

In step 803, the method further includes circulating the iontophoretic solution utilizing the delivery system, applying, during a predefined period of time and using an electrode device, a current to the delivery system. This delivery of current results in a therapeutically effective dose of the therapeutic substance being delivered via the delivery interface to a target area below the tissue surface. The circulating of the iontophoretic solution serves to replenish the concentration of the ions of the therapeutic substance within the nasal cavity during the predefined period of time to enhance amounts of the therapeutic substance that are delivered to the target area.

While steps 802-803 are shown separately in FIG. 8, these generally occur simultaneously. However, these steps do not have to be a complete overlap, i.e., step 802 can begin before step 803 begins (and vice versa) and step 802 can end before step 803 ends (and vice versa).

The circulating of the iontophoretic solution can be continuous or periodic. The average flow for this circulating can be such that the iontophoretic solution is replenished between 15 seconds to 1 minute, such as every 30 seconds.

The delivery of current can be by a profile that includes a ramp-up period (in which the current is ramped-up), a steady-state period (in which the current is maintained a constant amount), and a ramp-down period (in which the current is ramped-down). In some embodiments, the circulating of the iontophoretic solution occurs during the steady state period, but does not occur during the ramp-up and ramp-down periods.

In some embodiments, the periods of circulation are varied, such as shorter periods of circulation during the steady-state period (as compared to the ramp-up period and/or the ramp-down period).

After steps 801-803 are complete, in step 804, the method includes removing the delivery system from the nasal cavity. The delivery system can be removed by using a retrieval device, which can be a single component or mechanism such as, for example, a flexible shaft, beam, bar, wire, etc. The retrieval device can be capable of being coupled and uncoupled to the delivery system for removal of the delivery system. In some embodiments, the retrieval device is the same device as the deployment device.

Optionally, in step 805, this method can further include performing a test to determine the efficacy of the therapeutic substance. For example, when the iontophoretic solution includes ions of an anesthetics (such as lidocaine, benzocaine, procaine, amethocaine, cocaine, tetracaine, prilocaine, bupivicaine, levobupivacaine, ropivacaine, mepivacaine, dibucaine, etidocaine, etc.), a test can be performed to determine whether the patient has been properly anesthetized.

The therapeutic substance can be any suitable substance or combination of substances, in any suitable dosage form or combination of dosage forms. Non-limiting examples include analgesics (e.g., non-steroidal anti-inflammatory drugs (NSAIDs) like acetaminophen, COX-2 inhibitors, opioids, flupirtine, cannabinoids, capsaicinoids, etc.), anesthetics (e.g., lidocaine, benzocaine, procaine, amethocaine, cocaine, tetracaine, prilocaine, bupivicaine, levobupivacaine, ropivacaine, mepivacaine, dibucaine, etidocaine, etc.), anti-inflammation (e.g., NSAIDs like aspirin, ibuprofen, and naproxen, peptides, steroids or glucocorticosteroids like betamethasone, dexamethasone, etc.), antibiotics (e.g., ciprofloxacin, ciprofloxacin otic suspension, amoxicillin, amoxicillin-clavulanate, beta lactamase inhibitor, etc.), antivirals, antifungals, antiparasitics, decongestants (e.g., ephedrine, levomethamphetamine, naphazoline, oxymetazoline, phenylephrine, phenylpropanolamine, propylhexedrine, synephrine, tetrahydrozoline, xylometazoline, pseudoephedrine, tramazoline, etc.), mucokinetics (e.g., mucolytics like acetylcysteine, expectorants like guaifenesin, surfactants, etc.), antihistamines, antioxidants, immunosuppressive agents, and dissociatives (e.g., NMDA receptor antagonists like gacyclidine, K-opioid receptor agonists, etc.), steroids, sedatives, hypnotics, anticholinergics, antiemetics, antiepiletics, and combinations thereof.

The present invention is advantageous in that it provides for the delivery of the therapeutic substance quicker, and/or with less current being applied to the patient, which makes the process less uncomfortable for the patient. For instance, in the case of active ingredients lidocaine and epinephrine, this more rapidly anesthetizes the patient, which further lessens discomfort for the patient.

Another advantage of the present invention is that it utilizes existing pharmaceutical drugs instead of having to develop a new pharmaceutical drug from scratch.

While embodiments of the invention have been shown and described, modifications thereof can be made by one skilled in the art without departing from the spirit and teachings of the invention. The embodiments described and the examples provided herein are exemplary only and are not intended to be limiting. Many variations and modifications of the invention disclosed herein are possible and are within the scope of the invention. The scope of protection is not limited by the description set out above, but is only limited by the claims which follow, that scope including all equivalents of the subject matter of the claims.

The disclosures of all patents, patent applications, and publications cited herein are hereby incorporated herein by reference in their entirety, to the extent that they provide exemplary, procedural, or other details supplementary to those set forth herein.

Amounts and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of approximately 1 to approximately 4.5 should be interpreted to include not only the explicitly recited limits of 1 to approximately 4.5, but also to include individual numerals such as 2, 3, 4, and sub-ranges such as 1 to 3, 2 to 4, etc. The same principle applies to ranges reciting only one numerical value, such as “less than approximately 4.5,” which should be interpreted to include all of the above-recited values and ranges. Further, such an interpretation should apply regardless of the breadth of the range or the characteristic being described.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a” and “an” mean “one or more” when used in this application, including the claims.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that can vary depending upon the desired properties sought to be obtained by the presently disclosed subject matter.

As used herein, the term “about” and “substantially” when referring to a value or to an amount of mass, weight, time, volume, concentration or percentage is meant to encompass variations of the following: in some embodiments ±20%, in some embodiments ±10%, in some embodiments ±5%, in some embodiments ±1%, in some embodiments ±0.5%, and in some embodiments ±0.1%, each with respect to or from the specified amount, as such variations are appropriate to perform the disclosed method.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

What is claimed is:

1. A system comprising:
(a) a reservoir structure operable for receiving an iontophoretic solution comprising ions of a therapeutic substance;
(b) a delivery system comprising a delivery interface configured to be placed in operative apposition with a first side of a tissue surface within a nasal cavity of a subject, wherein
(i) the delivery system is configured for circulating the iontophoretic solution to the reservoir structure over a predefined period of time;
(c) an electrode device comprising a proximal portion and a distal portion, wherein
(i) the electrode device configured for the proximal portion to be disposed outside of the nasal cavity while the distal portion extends into the nasal cavity into engagement with a portion of the reservoir structure,
(ii) the electrode device further configured for applying a current to the reservoir structure such that an amount of the ions of the therapeutic substance is delivered to a target area of the subject on a second side of the tissue surface,
(iii) the reservoir structure configured to maintain the delivery interface against the first side of the tissue surface for at least the predefined period of time during which current is being applied by the electrode device to the reservoir structure, and
(iv) the circulating of the iontophoretic solution replenishes concentration of the ions of the therapeutic substance within the reservoir structure during the predefined period of time to enhance amounts of the therapeutic substance that are delivered to the target area.

2. The system of claim 1, wherein the delivery system is configured for circulating the iontophoretic solution having a continuous flow of iontophoretic solution.

3. The system of claim 1, wherein the delivery system is configured for circulating the iontophoretic solution having a periodic flow of iontophoretic solution.

4. The system of claim 1, wherein the delivery system is configured for circulating the iontophoretic solution at an average flow rate that replaces the iontophoretic solution in the reservoir structure in a period between 15 seconds and 1 minute.

5. The system of claim 1, wherein:
(a) the delivery system is configured to provide for the iontophoretic solution to flow into the reservoir structure at or nearby the tissue surface; and
(b) the delivery system is configured to provide for the iontophoretic solution to flow away from the reservoir structure at or nearby the distal portion of the electrode device.

6. The system of claim 1, wherein the electrode device is configured for applying the current utilizing a current profile having a ramp-up period, a steady state period, and a ramp-down period.

7. The system of claim 6, wherein the delivery system is configured for circulating the iontophoretic solution during the steady state period and for not circulating the iontophoretic solution during the ramp-up period and during the ramp-down period.

8. The system of claim 6, wherein
(a) the delivery system is configured for circulating the iontophoretic solution during the ramp-up period at an average ramp-up period flowrate;
(b) the delivery system is configured for circulating the iontophoretic solution during the steady state period at an average steady state period flow rate; and
(c) the average steady state period flow rate is greater than the average ramp-up period flowrate.

9. The system of claim 1 further comprises a deployment device that is capable of being coupled and uncoupled to the delivery system, wherein the deployment device is operable for placing the delivery system within the nasal cavity.

10. The system of claim 1 further comprises a retrieval device that is capable of being coupled and uncoupled to the delivery system, wherein the retrieval device is operable for removing the delivery system from the nasal cavity.

11. The system of claim 1 further comprises a deployment/retrieval device that is capable of being coupled and uncoupled to the delivery system, wherein the deployment/retrieval device is operable for placing the delivery system within the nasal cavity and is further operable for removing the delivery system from the nasal cavity.

12. The system of claim 1, wherein the therapeutic substance is selected from a group consisting of analgesics, anesthetics, anti-inflammatoires, antibiotics, antivirals, antifungals, antiparasitics, decongestants, mucokinetics, antihistamines, antioxidants, immunosuppressive agents, dissociatives, steroids, sedatives, hypnotics, anticholinergics, antiemetics, antiepiletics, non-steroidal anti-inflammatory drugs, and combinations thereof.

13. The system of claim 1, wherein the therapeutic substance is a steroid.

14. The system of claim 1, wherein the iontophoretic solution comprises betamethasone ions.

15. The system of claim 1, wherein the therapeutic substance is an anesthetic.

16. The system of claim 1, wherein the iontophoretic solution comprises lidocaine ions, epinephrine ions, or a combination thereof.

17. A method comprising:
(a) disposing a delivery system comprising a delivery interface in a nasal cavity of a subject such that the delivery interface is in operative apposition with a tissue surface;
(b) utilizing the delivery system to circulate an iontophoretic solution comprising ions of a therapeutic substance into the nasal cavity;
(c) while circulating the iontophoretic solution utilizing the delivery system, applying, during a predefined period of time and using an electrode device, a current to the delivery system such that a therapeutically effective dose of the therapeutic substance is delivered via the delivery interface to a target area below the tissue surface, wherein
(i) the circulating of the iontophoretic solution replenishes concentration of the ions of the therapeutic substance within the nasal cavity during the predefined period of time to enhance amounts of the therapeutic substance that are delivered to the target area.

18. The method of claim 17, wherein the step of circulating the iontophoretic solution is a continuous flow of iontophoretic solution.

19. The method of claim 17, wherein the step of circulating the iontophoretic solution is a periodic flow of iontophoretic solution.

20. The method of claim 17, wherein the step of circulating the iontophoretic solution is at an average flow rate that replaces the iontophoretic solution in the nasal cavity in a period between 15 seconds and 1 minute.

21. The method of claim 17, wherein the step of circulating the iontophoretic solution comprises:
(a) flowing the iontophoretic solution into the nasal cavity at or nearby the tissue surface; and
(b) flowing the iontophoretic solution at or nearby the electrode device away from the nasal cavity.

22. The method of claim 17, wherein the step of applying the current to the delivery system comprises applying the current utilizing a current profile having a ramp-up period, a steady state period, and a ramp-down period.

23. The method of claim 22, wherein the step of circulating the iontophoretic solution comprises circulating the iontophoretic solution during the steady state period and not circulating the iontophoretic solution during the ramp-up period and during the ramp-down period.

24. The method of claim 22, wherein
(a) the step of circulating the iontophoretic solution comprises circulating the iontophoretic solution during the ramp-up period at an average ramp-up period flowrate;
(b) the step of circulating the iontophoretic solution comprises circulating the iontophoretic solution during the steady state period at an average steady state period flow rate; and
(c) the average steady state period flow rate is greater than the average ramp-up period flowrate.

25. The method of claim 17 further comprising removing the delivery system from the nasal cavity.

26. The method of claim 25 further comprising performing a test to determine efficacy of the therapeutic substance delivered to the target area.

27. The method of claim 17, wherein the therapeutic substance is selected from a group consisting of analgesics, anesthetics, anti-inflammatoires, antibiotics, antivirals, antifungals, antiparasitics, decongestants, mucokinetics, antihistamines, antioxidants, immunosuppressive agents, dissociatives, steroids, sedatives, hypnotics, anticholinergics, antiemetics, antiepiletics, non-steroidal anti-inflammatory drugs, and combinations thereof.

28. The method of claim 17, wherein the therapeutic substance is a steroid.

29. The method of claim 17, wherein the iontophoretic solution comprises betamethasone ions.

30. The method of claim 17, wherein the therapeutic substance is an anesthetic.

31. The method of claim 17, wherein the iontophoretic solution comprises lidocaine ions, epinephrine ions, or a combination thereof.

32. A system comprising:
(a) a reservoir structure operable for receiving an iontophoretic solution comprising ions of a therapeutic substance;
(b) a delivery system comprising a delivery interface configured to be placed in operative apposition with a first side of a tissue surface within an interior region of a subject, wherein
(i) the delivery system is configured for circulating the iontophoretic solution to the reservoir structure over a predefined period of time:
(c) an electrode device comprising a proximal portion and a distal portion, wherein
(i) the electrode device configured for the proximal portion to be disposed outside of the interior region while the distal portion extends into the interior region into engagement with a portion of the reservoir structure,
(ii) the electrode device further configured for applying a current to the reservoir structure such that an amount of the ions of the therapeutic substance is delivered to a target area of the subject on a second side of the tissue surface,
(iii) the reservoir structure configured to maintain the delivery interface against the first side of the tissue surface for at least the predefined period of time during which current is being applied by the electrode device to the reservoir structure, and
(iv) the circulating of the iontophoretic solution replenishes concentration of the ions of the therapeutic substance within the reservoir structure during the predefined period of time to enhance amounts of the therapeutic substance that are delivered to the target area.

33. A method comprising:
(a) disposing a delivery system comprising a delivery interface in an interior region of a subject such that the delivery interface is in operative apposition with a tissue surface;
(b) utilizing the delivery system to circulate an iontophoretic solution comprising ions of a therapeutic substance into the interior region;
(c) while circulating the iontophoretic solution utilizing the delivery system, applying, during a predefined period of time and using an electrode device, a current to the delivery system such that a therapeutically effective dose of the therapeutic substance is delivered via the delivery interface to a target area below the tissue surface, wherein
(i) the circulating of the iontophoretic solution replenishes concentration of the ions of the therapeutic substance within the interior region during the predefined period of time to enhance amounts of the therapeutic substance that are delivered to the target area.
